(19) United States
(12) Patent Application Publication
(10) Pub. No.: US 2021/0208383 A1
(43) Pub. Date: Jul. 8, 2021

(54) LIGHT SOURCE CONTROL DEVICE, ENDOSCOPE SYSTEM, AND LIGHT QUANTITY CONTROL METHOD

(71) Applicant: OLYMPUS CORPORATION, Tokyo (JP)

(72) Inventor: Ryuichi YAMAZAKI, Tokyo (JP)

(73) Assignee: OLYMPUS CORPORATION, Tokyo (JP)

(21) Appl. No.: 17/193,111

(22) Filed: Mar. 5, 2021

Related U.S. Application Data
(63) Continuation of application No. PCT/JP2018/033038, filed on Sep. 6, 2018.

(51) Int. Cl. G02B 23/24; H05B 47/11

(52) U.S. Cl. CPC G02B 23/2461; A61B 1/0661; H05B 47/11; G02B 23/2484

(57) ABSTRACT

A light source control device for an endoscope includes one or more processors that perform processes. The processes include: (1) generating a light quantity control signal indicating excess or insufficiency of an illumination light quantity based on an imaging signal from an imaging element (2) determining whether the endoscope is left based on the light quantity control signal and whether the illumination light quantity is equal to or greater than a predetermined quantity; and (3) setting a control range that limits the illumination light quantity based on the light quantity control signal, wherein the setting the control range includes setting a second range having an upper limit lower than an upper limit of a first range as the control range, when the processors determine that the endoscope is left in a state in which the first range is set as the control range.

### CROSS REFERENCE TO RELATED APPLICATIONS

This is a Continuation Application of PCT Application No. PCT/JP2018/033038, filed Sep. 6, 2018, which was not published under PCT Article 21(2) in English.

### BACKGROUND OF THE INVENTION

### Field of the Invention

The disclosure herein relates to a light source control device, an endoscope system, and a light quantity control method.

### Description of the Related Art

An endoscope system allowing early detection and early treatment of lesions has been more increasingly used in the medical field in recent years.

A conventional endoscope system has a light quantity adjustment function of automatically adjusting an illumination light quantity to be supplied to an endoscope. The light quantity adjustment function is to make luminance of an image obtained by the endoscope close to target luminance or to maintain the luminance of the image.

In endoscopy, in some cases, the endoscope is hooked on a scope hanger and temporarily left, keeping an illumination function turned on. In this state, since the illumination light is emitted to the floor surface, a distance from the endoscope to an illumination target surface generally increases as compared with a state in which the endoscope is inserted into a body cavity. Therefore, a light quantity of reflected light from the illumination target surface to be introduced into an imaging element of the endoscope is reduced, which causes decrease in luminance of the image.

Accordingly, in the state in which the endoscope is left, the light quantity adjustment function serves to increase the illumination light quantity to increase the luminance of the image, but the luminance of the image cannot be sufficiently increased even when the illumination light quantity is increased. As a result, the illumination light quantity is continuously increased to an upper limit value, and after reaching the upper limit value, the illumination light quantity is maintained as it is. When the illumination light quantity is maintained at the upper limit value for a long period of time, a distal end of the endoscope reaches a high temperature, resulting in the failure of the endoscope, the deterioration of image quality, and the like.

Thus, the light quantity adjustment function of the conventional endoscope system has a technical issue in that the appropriate light quantity control is not performed in the state in which the endoscope is left outside the body cavity.

A technology relating to the above-described technical issue is disclosed in, for example, Japanese Patent Laid-Open No. 2006-334076 and International Publication No. WO 2011/102200. Japanese Patent Laid-Open No. 2006-334076 discloses a technology for reducing the light quantity when an image signal is not changed for a predetermined period of time. International Publication No. WO 2011/102200 discloses a technology for setting the upper limit value of the light quantity of the emission light to a small value when the endoscope is determined to be in a standby state in which an insertion portion of the endoscope is outside the body cavity.

### SUMMARY OF THE INVENTION

A light source control device according to one aspect of the present invention is a light source control device for an endoscope, the light source control device including one or more processors that perform processes. The processes include: (1) generating a light quantity control signal indicating excess or insufficiency of an illumination light quantity supplied from the light source control device to the endoscope based on an imaging signal from an imaging element of the endoscope; (2) determining whether the endoscope is left based on the light quantity control signal and whether the illumination light quantity is equal to or greater than a predetermined quantity; and (3) setting a control range that limits the illumination light quantity to be supplied from the light source control device to the endoscope based on the light quantity control signal, wherein the setting the control range includes setting a second range having an upper limit lower than an upper limit of a first range as the control range, when the one or more processors determines that the endoscope is left in a state in which the first range is set as the control range.

A light source control device according to another aspect of the present invention is a light source control device for an endoscope, the light source control device including one or more processors that perform the following processes. The processes include: (1) generating a light quantity control signal indicating excess or insufficiency of an illumination light quantity supplied from the light source control device to the endoscope based on an imaging signal from an imaging element of the endoscope; (2) determining whether the endoscope is left based on the light quantity control signal and whether the illumination light quantity is equal to or greater than a predetermined quantity; and (3) setting a control range that limits the illumination light quantity to be supplied from the light source control device to the endoscope based on the light quantity control signal, wherein the setting the control range includes setting a first range having an upper limit higher than an upper limit of a second range as the control range, when the one or more processors determines that the endoscope is not left in a state in which the second range is set as the control range.

An endoscope system according to one aspect of the present invention includes: an endoscope; a light source control device including one or more processors that perform processes; and a display device. The processes include: (1) generating a light quantity control signal indicating excess or insufficiency of an illumination light quantity supplied from the light source control device to the endoscope based on an imaging signal from an imaging element of the endoscope; (2) determining whether the endoscope is left based on the light quantity control signal and whether the illumination light quantity is equal to or greater than a predetermined quantity; and (3) setting a control range that limits the illumination light quantity to be supplied from the light source control device to the endoscope based on the light quantity control signal, wherein the setting the control range includes setting a second range having an upper limit lower than an upper limit of a first range as the control range, when the one or more processors determines that the endoscope is left in a state in which the first range is set as the control range. The display device displays a previous notification screen for previously notifying of a change of the control range from the first range to the second range or a screen showing that the illumination light quantity is suppressed during a period in which the second range is set.

A light quantity control method according to one aspect of the present invention is a light quantity control method for a light source control device for an endoscope, the method includes: generating a light quantity control signal indicating excess or insufficiency of an illumination light quantity supplied from the light source control device to the endoscope based on an imaging signal from an imaging element of the endoscope; determining whether the endoscope is left based on the light quantity control signal and whether the illumination light quantity is equal to or greater than a predetermined quantity; setting a control range that limits the illumination light quantity to be supplied from the light source control device to the endoscope based on the light quantity control signal; and setting a second range having an upper limit lower than an upper limit of a first range as the control range, when it is determined that the endoscope is left in a state in which the first range is set as the control range.

### BRIEF DESCRIPTION OF THE DRAWINGS

FIG. 1 is a diagram illustrating a configuration of an endoscope system 1 according to a first embodiment

FIG. 2 is a graph showing a control range of an illumination light quantity.

FIG. 3 illustrates an example of a flowchart of a light quantity control process performed by the endoscope system 1.

FIG. 4 illustrates an example of a flowchart of a light quantity control signal generation process.

FIG. 5 illustrates an example of a flowchart of a first leaving state determination process.

FIG. 6 illustrates an example of a flowchart of a suppression determination process.

FIG. 7 illustrates an example of a flowchart of a second leaving state determination process.

FIG. 8 illustrates an example of a flowchart of a release determination process.

FIG. 9 is a diagram illustrating an example of a suppression previous notification display screen.

FIG. 10 is a diagram illustrating an example of a suppression display screen.

FIG. 11 is an external view of the endoscope system 1.

FIG. 12 is a diagram illustrating a configuration of a light source device 30a according to a modification example.

FIG. 13 is a diagram illustrating a configuration of an endoscope system 2 according to a second embodiment.

FIG. 14 is an example of a flowchart of a light quantity control process performed by the endoscope system 2.

FIG. 15 is another example of a flowchart of a light quantity control process performed by the endoscope system 2.

FIG. 16 is a diagram illustrating a configuration of an endoscope system 3 according to a third embodiment.

FIG. 17 is a diagram illustrating a configuration of an endoscope system 4 according to a fourth embodiment.

### DETAILED DESCRIPTION OF THE PREFERRED EMBODIMENTS

In the technologies of the above-described patent documents, it is difficult to correctly recognize the state in which the endoscope is left outside the body cavity. For example, in Japanese Patent Laid-Open No. 2006-334076, the state of the endoscope is determined according to presence or absence of a change in the image signal. Therefore, even when the endoscope is inserted into the body cavity, if there is no change in the image, the light quantity is suppressed. In International Publication No. WO 2011/102200, when the light quantity of the emission light reaches the upper limit value and is maintained at the upper limit value for a predetermined period of time, the endoscope is determined to be in the standby state. Therefore, even when the endoscope is inserted into the body cavity, in the case where an object is continuously observed while being irradiated with light of the upper limit quantity, the light quantity is suppressed.

In view of the above-described circumstances, embodiments of the present invention will be described below.

### First Embodiment

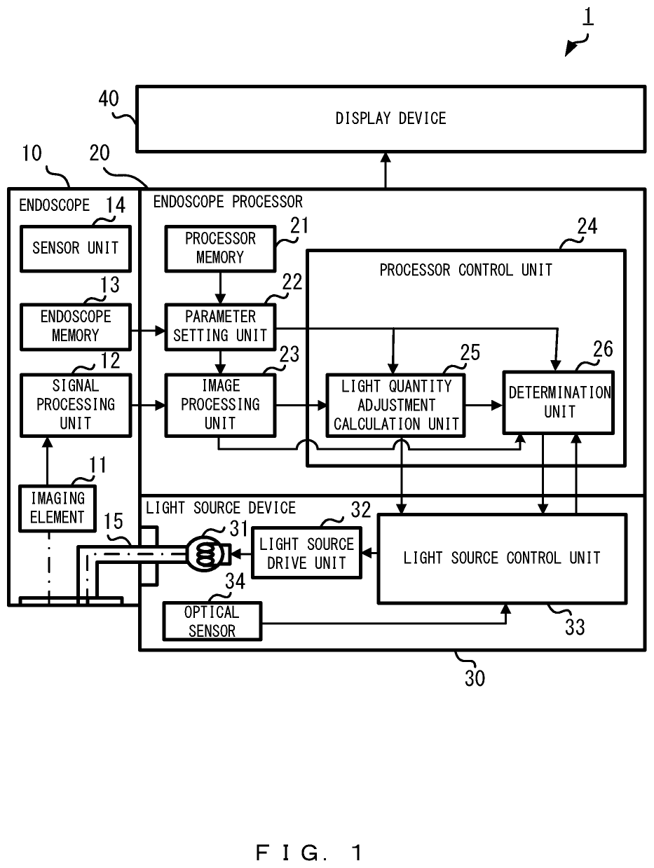

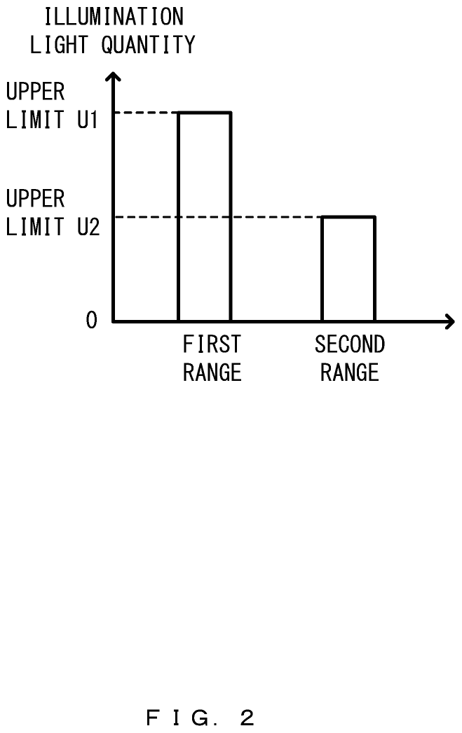

FIG. 1 is a diagram illustrating a configuration of an endoscope system 1 according to the present embodiment. FIG. 2 is a graph showing a control range of an illumination light quantity. The endoscope system 1 is a medical endoscope system provided with a flexible endoscope, and includes an endoscope 10, an endoscope processor 20, a light source device 30, and a display device 40, as illustrated in FIG. 1. Note that the endoscope processor 20 and the light source device 30 are herein collectively referred to as a light source control device for an endoscope.

In the light source control device or the endoscope system 1, appropriate light quantity control is performed in response to the state of the endoscope 10. Specifically, the light source control device or the endoscope system 1 determines the state of the endoscope 10 at least based on a light quantity control signal which will be described later, and switches, in response to the state of the endoscope 10, an illumination mode between a normal illumination mode in which the illumination light quantity to be supplied from the light source device 30 to the endoscope 10 is controlled within a first range and a suppression illumination mode in which the illumination light quantity is controlled within a second range, as shown in FIG. 2. Note that the second range has an upper limit U2 lower than an upper limit U1 of the first range, and the first range has the upper limit U1 higher than the upper limit U2 of the second range. Note that the upper limit U2 is, for example, a half of the upper limit U1. In this way, the appropriate light quantity control in response to the state of the endoscope 10 can be achieved.

First, a configuration of the light source control device or the endoscope system 1 will be described with reference to FIGS. 1 and 2.

The endoscope 10 is a flexible endoscope that is used for observation and diagnosis in each region of trachea and bronchus, for example. The endoscope 10 includes an insertion portion to be inserted into a specimen, an operation portion to be operated by an operator, a universal cord portion extending from the operation portion, and a connector portion provided on an end of the universal cord portion. The endoscope 10 outputs, to the endoscope processor 20, an imaging signal generated by capturing the specimen in a state in which the insertion portion is inserted into the body cavity as the specimen.

More specifically, the endoscope 10 includes an imaging element 11 and a light guide 15. The endoscope 10 may further include a signal processing unit 12, an endoscope memory 13, and a sensor unit 14.

The imaging element 11 includes, for example, a two-dimensional image sensor such as a charge coupled device (CCD) image sensor, and a complementary metal oxide semiconductor (CMOS) image sensor. The imaging element 11 is provided in the insertion portion, receives light from the specimen on a light-receiving surface via an optical system (not illustrated), and generates the imaging signal of the specimen by converting the received light into an electric signal.

The signal processing unit 12 is a circuit that processes the imaging signal. The signal processing unit 12 performs a predetermined process (a noise removal process, a clamp process) on the imaging signal which is an analog signal generated by the imaging element 11. Furthermore, the signal processing unit 12 performs analog-to-digital conversion, and outputs, to the endoscope processor 20, the imaging signal converted into digital data.

The endoscope memory 13 is a nonvolatile memory. Parameters corresponding to the endoscope 10 are stored in the endoscope memory 13. Specifically, identification information about the endoscope and various parameters for image processing are stored in the endoscope memory 13. Examples of the identification information about the endoscope include the identification information for identifying the endoscope 10, the identification information for identifying the model of the endoscope 10, and the like. Examples of the parameters for image processing include a parameter for white balance, a parameter for color correction, a parameter for aberration correction, and the like.

The sensor unit 14 includes a sensor that detects the operator's operation on the endoscope 10. For example, when the sensor unit 14 includes a pressure sensor, the sensor unit 14 can detect that the operator holds the endoscope 10. When the sensor unit 14 includes an acceleration sensor, the sensor unit 14 can detect that the operator has moved the endoscope 10. Furthermore, a button provided in the operation portion, or the like may be regarded as a component of the sensor unit 14, and the sensor unit 14 may detect a button operation by the operator.

The light guide 15 is disposed from the connector portion to the insertion portion via the universal cord portion and the operation portion, and guides, to the specimen, the illumination light supplied from the light source device 30.

The endoscope processor 20 is a control device that controls the operation of the endoscope system 1. The endoscope processor 20 causes the display device 40 to display an image of the specimen, based on the imaging signal output from the endoscope 10, for example. In addition, the endoscope processor 20 performs various processes. For example, the endoscope processor 20 performs a process relating to automatic light quantity control, and outputs, to the light source device 30, at least the light quantity control signal and the result of a leaving state determination process which will be described later. Note that, hereinafter, a configuration relating to the automatic light quantity control for controlling the illumination light quantity will be mainly described.

The endoscope processor 20 includes a processor memory 21, a parameter setting unit 22, an image processing unit 23, and a processor control unit 24. For example, the parameter setting unit 22, the image processing unit 23, and the processor control unit 24 (a light quantity adjustment calculation unit 25, a determination unit 26) may be configured using a general-purpose processor such as a CPU, or may be configured using a special-purpose processor such as ASIC or FPGA.

The processor memory 21 is a nonvolatile memory. Various parameters for image processing and control processing are stored in the processor memory 21. Examples of the parameters for control processing include a target value of the brightness of the image, parameters (an illumination light quantity, duration) used for the leaving state determination process, and a control range (a first range, a second range) of the illumination light quantity, which will be described later. A plurality of target values of the brightness may be included in the parameters for control processing. For example, the parameters for control processing may include five target values corresponding to five levels of brightness that can be selected by the operator through operation of the button provided in the operation portion of the endoscope 10, or the like, for example.

The parameter setting unit 22 outputs, to the image processing unit 23 and the processor control unit 24, the parameters and the identification information that are read from the processor memory 21 and the endoscope memory 13.

The image processing unit 23 performs an OB subtraction process, a WB correction process, a demosaicing process, a color matrix process, and the like on the imaging signal output from the signal processing unit 12, and outputs the processed imaging signal to the processor control unit 24. For example, in the OB subtraction process, an optical black (OB) value due to dark current of the imaging element 11 or the like is subtracted from a pixel value of each pixel calculated from the imaging signal. In the WB correction process, the white balance of the imaging signal is corrected by amplifying the pixel value of each color (for example, R, B) using the parameter (for example, an R gain, a B gain) for white balance read from the endoscope memory 13. In the demosaicing process, the data of color not included in a pixel is calculated by interpolating the data of the color included in the surrounding pixels, for each pixel included in the imaging signal. In the color matrix process, the color of the imaging signal is corrected by multiplying the demosaicing-processed imaging signal by a parameter (for example, a color matrix coefficient) for color correction that is read from the endoscope memory 13. In addition, in the image processing unit 23, an electronic zoom process, an edge enhancement process, a gamma correction process, or the like may be performed on the imaging signal.

The processor control unit 24 controls operation of an external device connected to the endoscope processor 20 by outputting the calculation result to the external device. For example, the processor control unit 24 includes the light quantity adjustment calculation unit 25, and the determination unit 26 as the configuration relating to the automatic light quantity control, and outputs, to the light source device 30, the light quantity control signal and a result of the leaving state determination process, which will be described later.

The light quantity adjustment calculation unit 25 generates the light quantity control signal at least based on the imaging signal, and outputs the generated signal to the light source device 30. The light quantity control signal is a signal indicating an excess or insufficiency of the illumination light quantity supplied from the light source device 30 to the endoscope 10. In the endoscope system 1, the light quantity control signal indicating that the illumination light quantity is insufficient operates as an instruction to increase (an Up instruction) the illumination light quantity to be issued to the light source device 30, and the light quantity control signal indicating that the illumination light quantity is too large operates as an instruction to decrease (a Down instruction) the illumination light quantity to be issued to the light source device 30, so that automatic light quantity adjustment is performed. Note that the light quantity control signal is also referred to as an “EE signal,” and may include not only the information about the excess or insufficiency but also the information about the degree of excess or insufficiency.

Specifically, the light quantity adjustment calculation unit 25 generates the light quantity control signal at least based on the evaluation value of the brightness of a image calculated from the imaging signal and the target value of the brightness of the image. More specifically, the light quantity adjustment calculation unit 25 may first calculate the evaluation value of the brightness of the image from the imaging signal output from the image processing unit 23. The evaluation value of the brightness of the image may be calculated based on a luminance signal included in the imaging signal, for example. Furthermore, the light quantity adjustment calculation unit 25 may acquire the target value of the brightness of the image. The light quantity adjustment calculation unit 25 may acquire the target value corresponding to a level of the brightness selected by the operator from the processor memory 21 via the parameter setting unit 22. The light quantity adjustment calculation unit 25 that has acquired the evaluation value and the targe value may generate the light quantity control signal based on the ratio between the evaluation value and the target value. For example, the light quantity control signal may be calculated as the “evaluation value/the target value” or the “target value/the evaluation value.” In this way, generating the light quantity control signal using the target value enables the light quantity control so that the brightness of the image approaches the target value.

The determination unit 26 determines whether the endoscope 10 is left, at least based on the light quantity control signal, and outputs the result of the leaving state determination process to the light source device 30. Specifically, the determination unit 26 determines whether the endoscope 10 is left at least based on the light quantity control signal and the information on the illumination light quantity, in the normal illumination mode, namely in the case where the first range is set as the control range. The information on the illumination light quantity may be the information on the illumination light quantity supplied from the light source device 30 to the endoscope 10, or may be the information on the illumination light quantity to be supplied from the light source device 30 to the endoscope 10. In any case, the information on the illumination light quantity is acquired from a light source control unit 33. Note that the light source control unit 33 may generate the information on the illumination light quantity supplied to the endoscope 10 based on, for example, the illumination light quantity measured by an optical sensor 34 which will be described later. The light source control unit 33 may generate the information on the illumination light quantity to be supplied to the endoscope 10 based on, for example, the illumination light quantity instructed to a light source drive unit 32 which will be described later.

More specifically, the determination unit 26 first determines, in the normal illumination mode, whether a predetermined state is maintained for a predetermined period of time or longer, at least based on the light quantity control signal and the information on the illumination light quantity. Then, when determined that the predetermined state is maintained for the predetermined period of time of longer, the determination unit 26 determines that the endoscope 10 is left, and otherwise, determines that the endoscope 10 is not left. Then, the determination unit 26 outputs the result of the leaving state determination process to the light source control unit 33. For example, the above-described predetermined state refers to a state in which the illumination light quantity is equal to or greater than a predetermined quantity and the light quantity control signal indicating the insufficiency of the illumination light quantity is generated. The predetermined light quantity refers to, for example, a light quantity corresponding to the upper limit of the first range shown in FIG. 2, and the predetermined period of time is, for example, 120 seconds.

Additionally, the determination unit 26 may determine whether the endoscope 10 is left according to the criteria different from those in the normal illumination mode, in the suppression illumination mode, namely in the case where the second range is set as the control range. More specifically, the determination unit 26 may determine that the endoscope 10 is left according to the criteria more stringent than those in the normal illumination mode in which the first range is set as the control range, in the suppression illumination mode in which the second range is set as the control range. In this way, setting the leaving state determination criteria in the suppression illumination mode more stringently than those in the normal illumination mode can prevent a situation that even when the operator operates the endoscope 10, the illumination mode is not immediately shifted to the normal illumination mode, whereby the illumination light quantity remains low without being recovered. This makes it possible to avoid the situation that the operator erroneously recognizes that the endoscope 10 has failed, when viewing the dark image.

For example, the determination unit 26 may determine whether the image processing unit 23 has detected a change in the imaging signal. When determining that the change has been detected, the determination unit 26 determine that the endoscope 10 is not left. For example, the presence or absence of the change in the imaging signal may be determined based on a motion vector calculated from the imaging signal, or may be determined based on the contrast of the image calculated from the imaging signal. The determination may be made based on the luminance of the image calculated from the imaging signal, or the evaluation value of the brightness of the image. Note that this is substantially the same as when the determination is made based on the change in the light quantity control signal. In addition, the determination unit 26 may determine whether the sensor unit 14 of the endoscope 10 has detected the operation on the endoscope 10, and determine that the endoscope 10 is not left when determining that the operation has been detected. Furthermore, in the same manner as the normal illumination mode, the determination unit 26 may determine whether the predetermined state is maintained for the predetermined period of time of longer, and determine that the endoscope 10 is not left when not determining that the predetermined state is maintained for the predetermined period of time. Note that the predetermined quantity in the suppression illumination mode may be different the predetermined quantity in the normal illumination mode. The predetermined quantity in the suppression illumination mode may be, for example, a light quantity corresponding to the upper limit of the second range shown in FIG. 2.

In other words, the criteria in the suppression illumination mode adopted more stringently than those in the normal illumination mode may be, for example, criteria that a plurality of number of determination processes larger than the number of determination processes performed in the normal illumination mode are performed, and it is determined that the endoscope 10 is not left unless it is determined that the endoscope 10 is left in all of the determination processes. The plurality of number of determination processes larger than the number of determination processes performed in the normal illumination mode may include at least one of the above-described plurality of number of determination processes such as a determination process relating to the change in the imaging signal, a determination process relating to the evaluation value of the brightness of the image, a determination process relating to the operation detection, and a determination process relating to the predetermined state and the predetermined period of time.

The light source device 30 is a device that supplies the illumination light to the endoscope 10, and performs the automatic light quantity control at least using the light quantity control signal and the result of the leaving state determination process that are acquired from the endoscope processor 20. The endoscope 10 is detachably attached to the light source device 30.

The light source device 30 includes a light source 31, the light source drive unit 32, and the light source control unit 33. The light source device 30 may further include the optical sensor 34.

The light source 31 is a light source that emits the illumination light to be supplied to the endoscope 10. Hereinafter, the description will be made of an example in which the light source 31 is a white light emitting diode (LED) light source, but the light source 31 is not limited to the LED light source, and may be a lamp light source such as a xenon lamp and a halogen lamp, or may be a laser light source. The light source 31 may include a plurality of LED light sources that emit the illumination light in different colors.

The light source drive unit 32 is a driver that drives the light source 31, and is, for example, an LED driver. The light source drive unit 32 drives the light source 31 according to a command value (for example, a current value, a voltage value) from the light source control unit 33. Note that the command value input from the light source control unit 33 is a value for indirectly indicating the illumination light quantity to be supplied to the endoscope 10. For example, when the light source 31 is an LED light source, the command value (current value) and the illumination light quantity have a substantially proportional relationship.

The light source control unit 33 performs the automatic light quantity control by controlling the illumination light quantity to be supplied from the light source device 30 to the endoscope 10 within the set control range at least based on the light quantity control signal. Specifically, when the light quantity control signal indicates the Up instruction, the light source control unit 33 controls to increase the illumination light quantity within the control range. Additionally, when the light quantity control signal indicates the Down instruction, the light source control unit 33 controls to decrease the illumination light quantity within the control range. Note that the light source control unit 33 may be configured using a general-purpose processor such as a CPU, or may be configured using a special-purpose processor such as ASIC or FPGA.

The light source control unit 33 sets any one of the first range and the second range shown in FIG. 2 as the control range in response to the state of the endoscope 10. In this way, the illumination mode is switched between the normal illumination mode and the suppression illumination mode in response to the state of the endoscope 10, and the appropriate light quantity control is performed.

Specifically, when the determination unit 26 determines that the endoscope 10 is left in a state in which the first range is set as the control range, the light source control unit 33 sets the second range as the control range, and switches the illumination mode from the normal illumination mode to the suppression illumination mode.

Furthermore, when the determination unit 26 determines that the endoscope 10 is not left in a state in which the second range is set as the control range, the light source control unit 33 sets the first range as the control range, and switches the illumination mode from the suppression illumination mode to the normal illumination mode.

The optical sensor 34 measures the quantity of the illumination light emitted from the light source 31, and outputs the measurement result to the light source control unit 33.

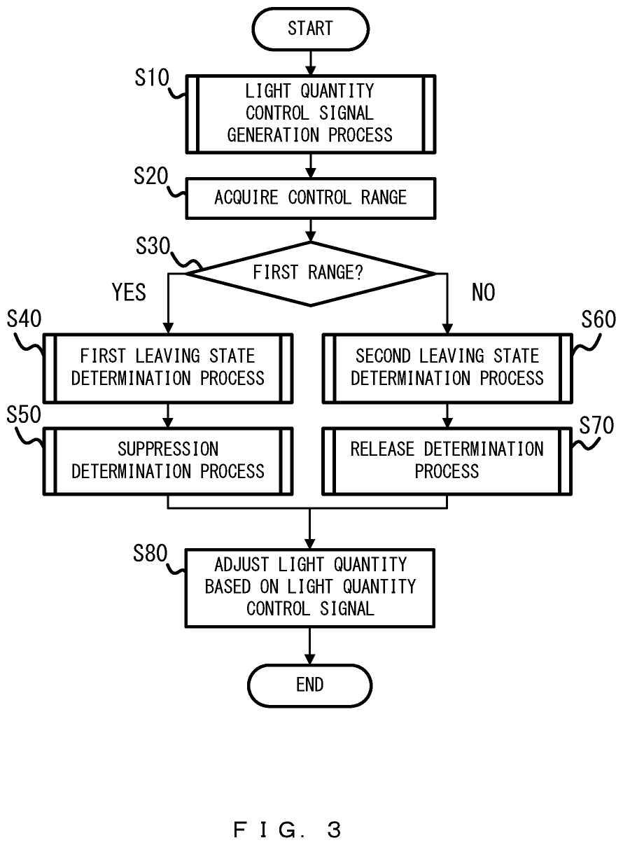

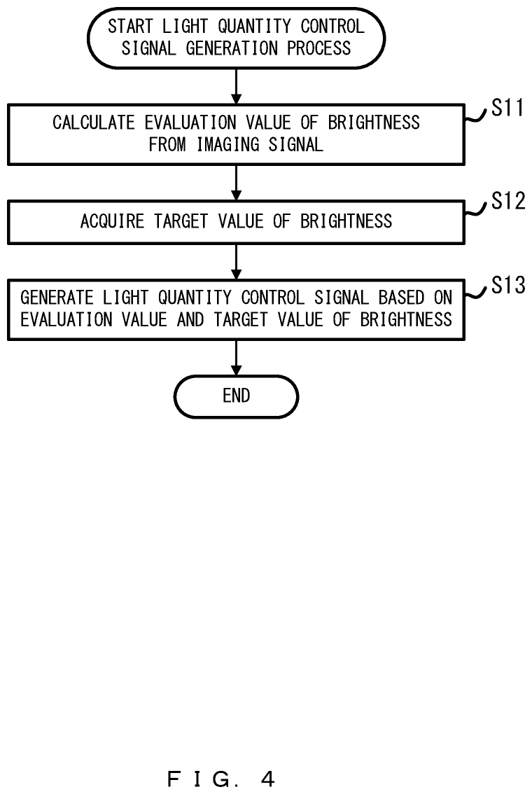

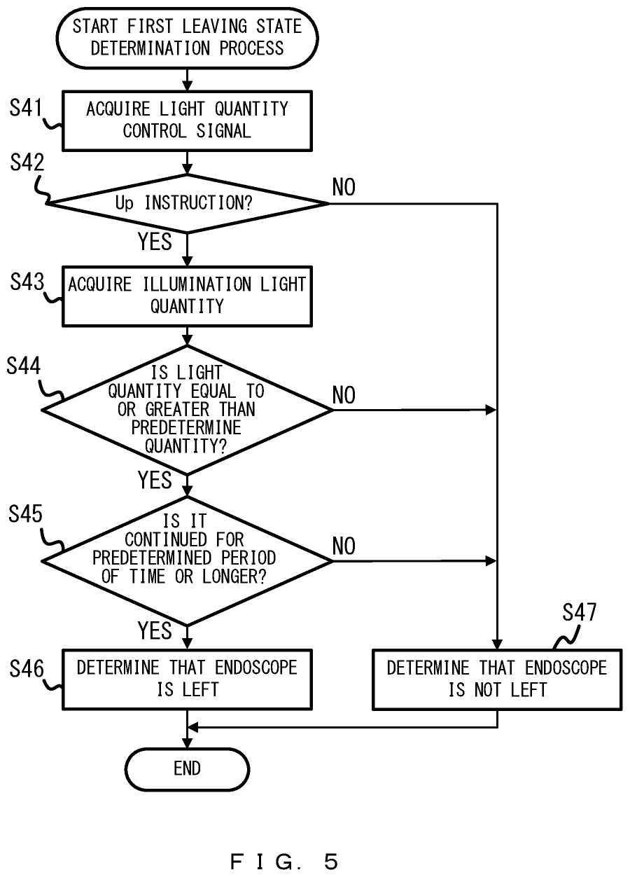

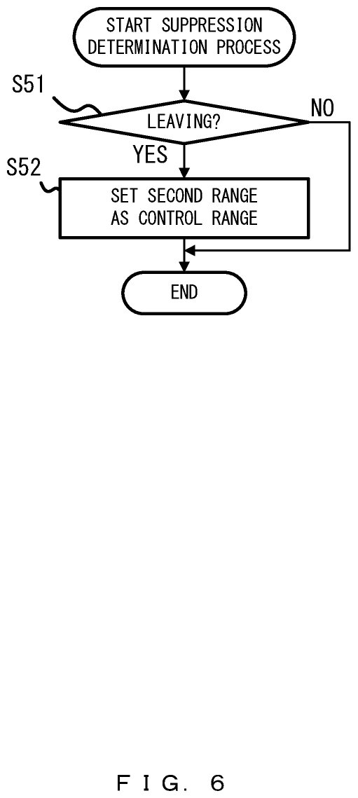

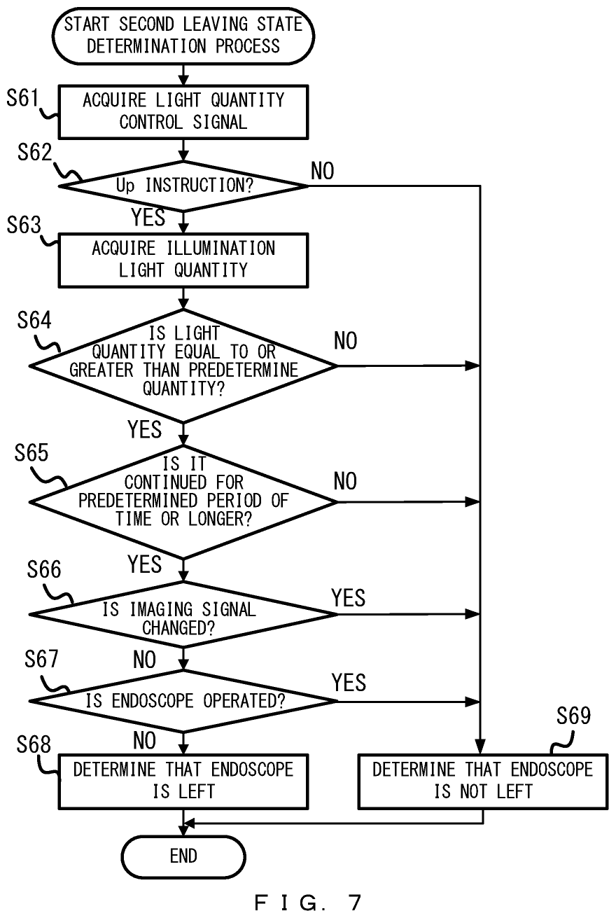

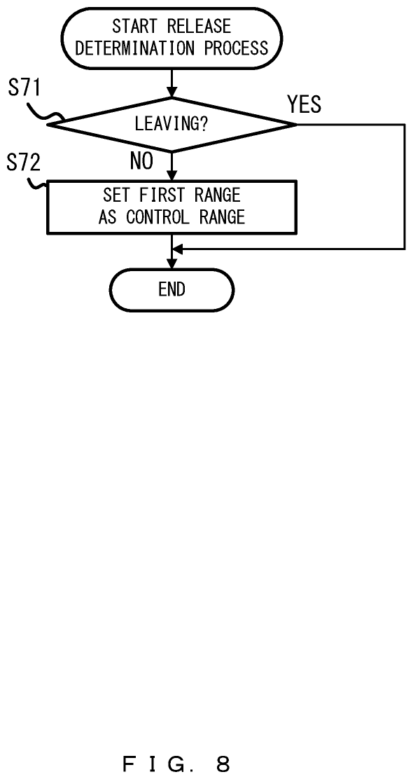

FIG. 3 illustrates an example of a flowchart of a light quantity control process performed by the endoscope system 1. FIG. 4 illustrates an example of a flowchart of a light quantity control signal generation process. FIG. 5 illustrates an example of a flowchart of a first leaving state determination process. FIG. 6 illustrates an example of a flowchart of a suppression determination process. FIG. 7 illustrates an example of a flowchart of a second leaving state determination process. FIG. 8 illustrates an example of a flowchart of a release determination process.

Hereinafter, the light quantity control method for the light source control device included in the endoscope system 1 will be specifically described with reference to FIGS. 3 to 8. In the endoscope system 1, when the automatic light quantity adjustment function of the endoscope system 1 is turned on, the light quantity control process illustrated in FIG. 3 is started.

When the light quantity control process is started, the endoscope processor 20 first performs the light quantity control signal generation process (step S10). When the light quantity control signal generation process illustrated in FIG. 4 is started, the light quantity adjustment calculation unit 25 calculates the evaluation value of the brightness from the imaging signal output from the image processing unit 23 (step S11). Furthermore, the light quantity adjustment calculation unit 25 acquires the target value of the brightness from the processor memory 21 via the parameter setting unit 22 (step S12). Finally, the light quantity adjustment calculation unit 25 generates the light quantity control signal based on the evaluation value calculated in step S11 and the target value acquired in step S12 (step S13), and outputs the generated light quantity control signal to the determination unit 26 and the light source control unit 33. The light quantity control signal is generated based on, for example, the ratio between the target value and the evaluation value.

Note that FIG. 4 illustrates an example in which step S12 is performed after step S11, but it is only required that steps S11 and S12 are performed before step S13. In other words, step S11 may be performed after step S12, or steps S11 and S12 may be performed in parallel.

When the light quantity control signal is generated, the endoscope processor 20 acquires the current setting of the control range (step S20), and determines whether the set control range is the first range (step S30). When the first range is set, the endoscope processor 20 perform the first leaving state determination process (step S40), and then the light source device 30 performs the suppression determination process (step S50). On the other hand, when the second range is set, the endoscope processor 20 performs the second leaving state determination process (step S60), and then the light source device 30 performs the release determination process (step S70).

When the first leaving state determination process illustrated in FIG. 5 is started, the determination unit 26 acquires the light quantity control signal from the light quantity adjustment calculation unit 25 (step S41), and determines whether the content of the acquired light quantity control signal indicates the Up instruction or that the illumination light quantity is insufficient (step S42). When not determining, in step S42, that the light quantity control signal indicates the Up instruction, the determination unit 26 determines that the endoscope 10 is not left (step S47), and ends the first leaving state determination process.

When determining, in step S42, that the light quantity control signal indicates the Up instruction, the determination unit 26 acquires the information on the illumination light quantity from the light source control unit 33 (step S43), and determines whether the illumination light quantity is equal to or greater than the predetermined quantity (step S44). Here, the information on the illumination light quantity may be the information on the illumination light quantity measured by the optical sensor 34, or may be the information on the illumination light quantity generated based on the command value output from the light source control unit 33 to the light source drive unit 32. Furthermore, it is desirable that the predetermined quantity is a light quantity corresponding to the upper limit of the first range. When not determining, in step S44, that the illumination light quantity is equal to or greater than the predetermined quantity, the determination unit 26 determines that the endoscope 10 is not left (step S47), and ends the first leaving state determination process.

When determining, in step S44, that the illumination light quantity is equal to or greater than the predetermined quantity, the determination unit 26 determines whether the state in which the light quantity control signal indicates the Up instruction and the illumination light quantity is equal to or greater than the predetermined quantity is continued for the predetermined period of time or longer (step S45). When not determining, in step S45, that the above-described state is continued for the predetermined period of time or longer, the determination unit 26 determines that the endoscope 10 is not left (step S47), and ends the first leaving state determination process.

When determining, in step S45, that the above-described state is continued for the predetermined period of time or longer, the determination unit 26 determines that the endoscope 10 is left (step S46), and ends the first leaving state determination process.

Note that, in FIG. 5, the processes are performed in order of steps S41 to S45, to determine that the endoscope 10 is left, but the order of the processes is not limited to the order illustrated in FIG. 5. The determination that the endoscope 10 is left can be made when the light quantity control signal indicates the Up instruction and the illumination light quantity is equal to or greater than the predetermined quantity, and the two conditions are continuously maintained for the predetermined period of time or longer. Therefore, the determination that the endoscope 10 is left may be made by performing the processes in order different from the order of the processes illustrated in FIG. 5.

When the first leaving state determination process is ended, the light source device 30 performs the suppression determination process (step S50). When the suppression determination process illustrated in FIG. 6 is started, and the determination result of the first leaving state determination process illustrated in FIG. 5 shows that “the endoscope 10 is not left” (No in step S51), the light source control unit 33 ends the suppression determination process.

When the determination result of the first leaving state determination process illustrated in FIG. 5 shows that “the endoscope 10 is left” (Yes in step S51), the light source control unit 33 sets the second range as the control range of the illumination light quantity (step S52), and ends the suppression determination process.

On the other hand, when the second leaving state determination process illustrated in FIG. 7 is started, the determination unit 26 acquires the light quantity control signal from the light quantity adjustment calculation unit 25 (step S61), and determines whether the content of the acquired light quantity control signal indicates the Up instruction or that the illumination light quantity is insufficient (step S62). When not determining, in step S62, that the light quantity control signal indicates the Up instruction, the determination unit 26 determines that the endoscope 10 is not left (step S69), and ends the second leaving state determination process.

When determining, in step S62, that the light quantity control signal indicates the Up instruction, the determination unit 26 acquires the information on the illumination light quantity from the light source control unit 33 (step S63), and determines whether the illumination light quantity is equal to or greater than the predetermined quantity (step S64). Here, the information on the illumination light quantity may be the information on the illumination light quantity measured by the optical sensor 34, or may be the information on the illumination light quantity generated based on the command value output from the light source control unit 33 to the light source drive unit 32. Furthermore, it is desirable that the predetermined quantity is a light quantity corresponding to the upper limit of the first range. When not determining, in step S64, that the illumination light quantity is equal to or greater than the predetermined quantity, the determination unit 26 determines that the endoscope 10 is not left (step S69), and ends the second leaving state determination process.

When determining, in step S64, that the illumination light quantity is equal to or greater than the predetermined quantity, the determination unit 26 determines whether the state in which the light quantity control signal indicates the Up instruction and the illumination light quantity is equal to or greater than the predetermined quantity is continued for the predetermined period of time or longer (step S65). When not determining, in step S65, that the above-described state is continued for the predetermined period of time or longer, the determination unit 26 determines that the endoscope 10 is not left (step S69), and ends the second leaving state determination process.

When determining, in step S65, that the above-described state is continued for the predetermined period of time or longer, the determination unit 26 further determines whether the image processing unit 23 has detected the change in the imaging signal (step S66). When determining, in step S66, that the change has been detected, the determination unit 26 determines that the endoscope 10 is not left (step S69), and ends the second leaving state determination process.

When not determining, in step S66, that the change in the imaging signal has been detected, the determination unit 26 further determines whether the sensor unit 14 has detected the operation on the endoscope 10 (step S67). When determining, in step S67, that the operation has been detected, the determination unit 26 determines that the endoscope 10 is not left (step S69), and ends the second leaving state determination process. When not determining, in step S67, that the operation has been detected, the determination unit 26 determines that the endoscope 10 is left (step S68), and ends the second leaving state determination process.

When the second leaving state determination process is ended, the light source device 30 performs the release determination process (step S70). When the release determination process illustrated in FIG. 8 is started, and the determination result of the second leaving state determination process illustrated in FIG. 7 shows that “the endoscope 10 is not left” (No in step S71), the light source control unit 33 sets the first range as the control range of the illumination light quantity (step S72), and ends the release determination process.

When the determination result of the second leaving state determination process illustrated in FIG. 7 shows that “the endoscope 10 is left” (Yes in step S71), the light source control unit 33 ends the release determination process.

When the suppression determination process or the release determination process is ended, the light source device 30 adjusts light quantity based on the light quantity control signal generated in step S10 (step S80). Here, the light source control unit 33 determines the illumination light quantity based on the light quantity control signal within the currently set control range, and outputs, to the light source drive unit 32, the command value corresponding to the determined illumination light quantity. In this way, the illumination light quantity corresponding to the command value is emitted from the light source 31, and the specimen is irradiated with the illumination light of the determined quantity via the endoscope 10.

As described above, the light source control device or the endoscope system 1 according to the present embodiment performs the leaving state determination process using the light quantity control signal. The situation that the image is not bright enough and the brightness is continuously insufficient for the target value for a predetermined period of time or longer even though the illumination light quantity has reached the upper limit cannot occur usually when the endoscope 10 is used in the body cavity. Such a situation is a unique situation that occurs when the endoscope 10 is left outside the body cavity, and can be detected for the first time by using the light quantity control signal. The light source control device or the endoscope system 1 according to the present embodiment can determine the leaving state with higher accuracy than the conventional endoscope system by performing the leaving state determination process using the light quantity control signal. This enables the appropriate light quantity control in response to the state of the endoscope.

Note that it is desirable that the illumination light quantity as reference for the leaving state determination is the light quantity corresponding to the upper limit of the first range. However, it can be the light quantity that is sufficient to obtain the bright image, and is not limited to the light quantity corresponding to the upper limit of the first range.

In addition, the light source device or the endoscope system 1 according to the present embodiment determines whether the endoscope 10 is in the leaving state, using different references between when the illumination mode is the normal illumination mode and when the illumination mode is the suppression illumination mode. More specifically, in the suppression illumination mode, the “leaving state” is determined more strictly than in the normal illumination mode. Therefore, the suppression of the control range is automatically released in the case where there is any suspicion that the endoscope 10 is not in the leaving state during operation in the suppression illumination mode. Accordingly, the light source control device or the endoscope system 1 according to the present embodiment can surely prevent the situation that the illumination light quantity is limited during use of the endoscope 10.

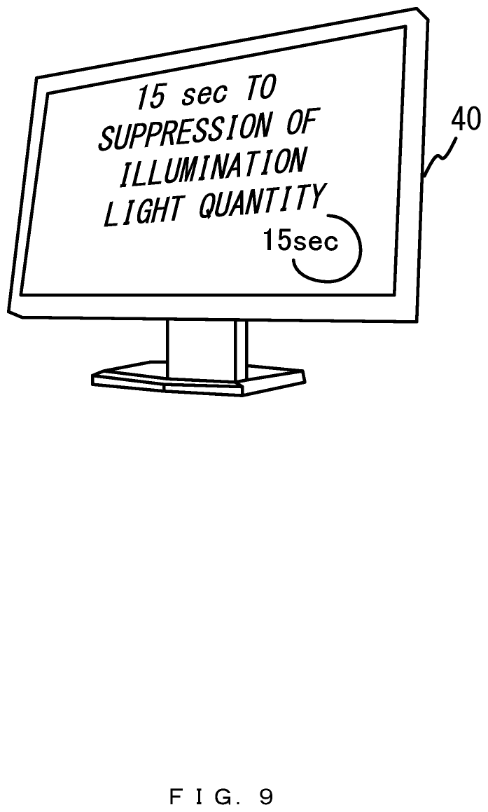

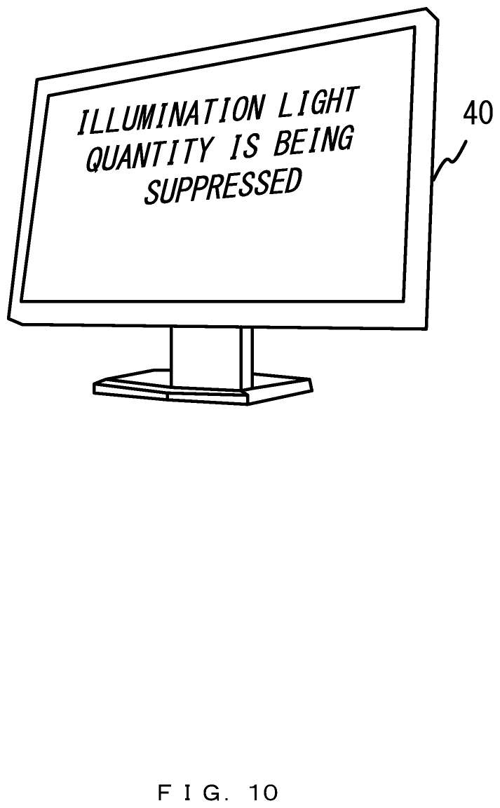

FIG. 9 is a diagram illustrating an example of a suppression previous notification display screen. FIG. 10 is a diagram illustrating an example of a suppression display screen. In the endoscope system 1 according to the present embodiment, a previous notification screen for previously notifying of the change of the control range may be displayed on the display device 40 before the control range is changed from the first range to the second range. It is desirable that a remaining time until the control range is changed is displayed on the previous notification display screen, as illustrated in FIG. 9. This can prevent the image from suddenly darkening without the operator being aware of it, which can avoid the situation that the operator suspects the failure of the device. Furthermore, in the endoscope system 1 according to the present embodiment, for example, the suppression display screen indicating that the illumination light quantity is suppressed may be displayed on the display device 40, during the period (suppression illumination mode) in which the second range is set as the control range, as illustrated in FIG. 10. In this way, the reason why the image is dark is indicated to the operator, which can avoid the situation that the operator suspects the failure of the device.

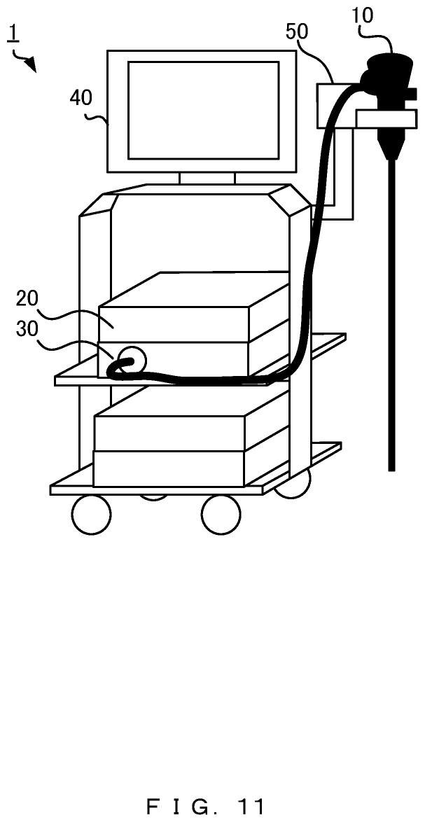

FIG. 11 is an external view of the endoscope system 1. As illustrated in FIG. 11, the endoscope system 1 according to the present embodiment may include an endoscope hanger 50, and may further include a sensor that detects that the endoscope 10 is hooked on the endoscope hanger 50. The endoscope processor 20 may be configured to detect the leaving state when the sensor detects that the endoscope 10 is hooked on the endoscope hanger 50.

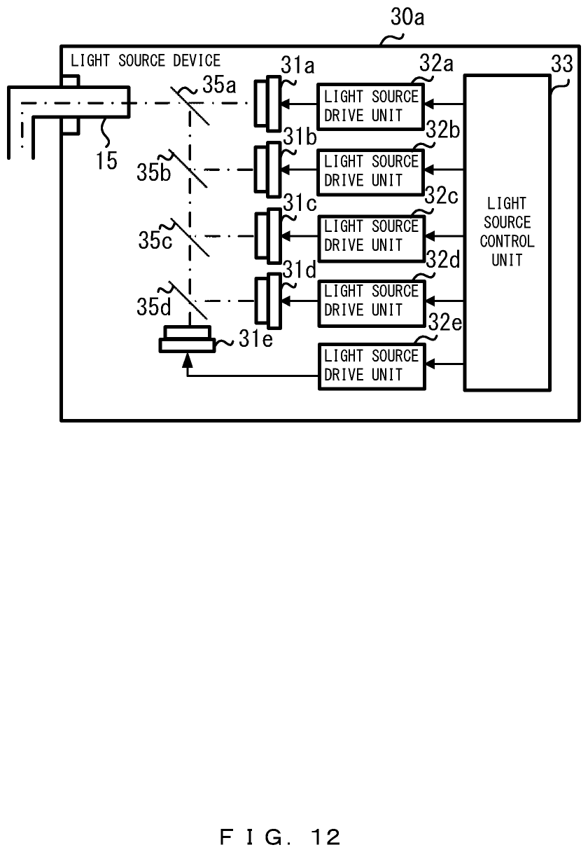

FIG. 12 is a diagram illustrating a configuration of a light source device 30a. The light source device 30a illustrated in FIG. 12 is a modification example of the light source device 30 included in the endoscope system 1, and the endoscope system 1 may include a light source device 30a instead of the light source device 30.

The light source device 30a includes a plurality of light sources (a light source 31a, a light source 31b, a light source 31c, a light source 31d, and a light source 31e) that emit illumination light in different wavelength regions. The plurality of light sources are, for example, LED light sources that emit the illumination light in the wavelength regions such as violet (V), blue (B), green (G), and red (R). The light source device 30a further includes a plurality of light source drive units (a light source drive unit 32a, a light source drive unit 32b, a light source drive unit 32c, a light source drive unit 32d, and a light source drive unit 32e) that drive a plurality of light sources, respectively. The illumination light emitted from the plurality of light sources is combined by a plurality of dichroic mirrors (a dichroic mirror 35a, a dichroic mirror 35b, a dichroic mirror 35c, and a dichroic mirror 35d), and then enters the light guide 15.

In the light source device 30a, a light source control unit 33 controls the illumination light quantity to be supplied from the light source device 30a to the endoscope 10 by outputting command values to the respective light source drive units. Note that the light source control unit 33 may cause all the five light sources to emit light when imaging using white light (WL1) is performed, for example, and may cause at least one of the five light sources to emit light when special light imaging (for example, NBI, AFI, and the like) is performed.

In the light source device 30a, the light source control unit 33 may maintain a light quantity ratio of the illumination light emitted from the plurality of light sources, between the case where the first range is set as the control range and the case where the second range is set as the control range. In this way, the color balance can be maintained by the illumination light emitted in the state in which the second range is set and the illumination light emitted in the state in which the first range is set. However, in the state in which the second range is set, the observation is not normally performed. Therefore, in the light source device 30a, the light source control unit 33 may suppress the light quantity to be supplied from the light source device 30 to the endoscope 10 by suppressing the illumination light quantity from a particular light source, when the second range is set as the control range.

### Second Embodiment

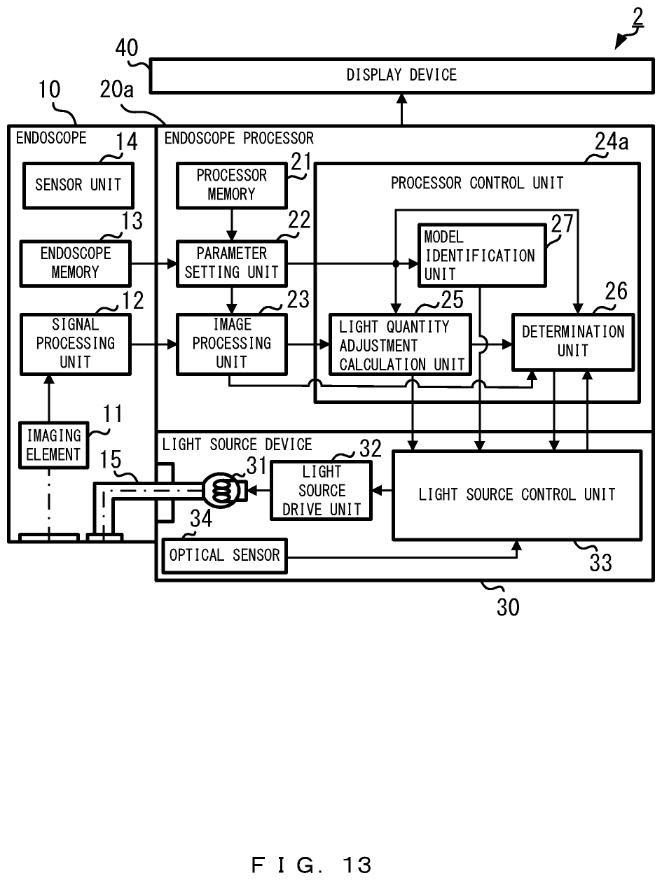

FIG. 13 is a diagram illustrating a configuration of an endoscope system 2 according to the present embodiment. The endoscope system 2 illustrated in FIG. 13 is different from the endoscope system 1 in that an endoscope processor 20a is provided instead of the endoscope processor 20. The other components are the same as those in the endoscope system 1.

The endoscope processor 20a is different from the endoscope processor 20 in that a processor control unit 24a is provided instead of the processor control unit 24. The processor control unit 24a is different from the processor control unit 24 in that a model identification unit 27 is provided in addition to the light quantity adjustment calculation unit 25 and the determination unit 26.

The model identification unit 27 is a circuit that identifies a model of the endoscope 10 connected to the light source device 30. The model identification unit 27 identifies the model of the endoscope 10 based on the information of the endoscope 10 read from the endoscope memory 13 via the parameter setting unit 22, more specifically, the model information of the endoscope 10.

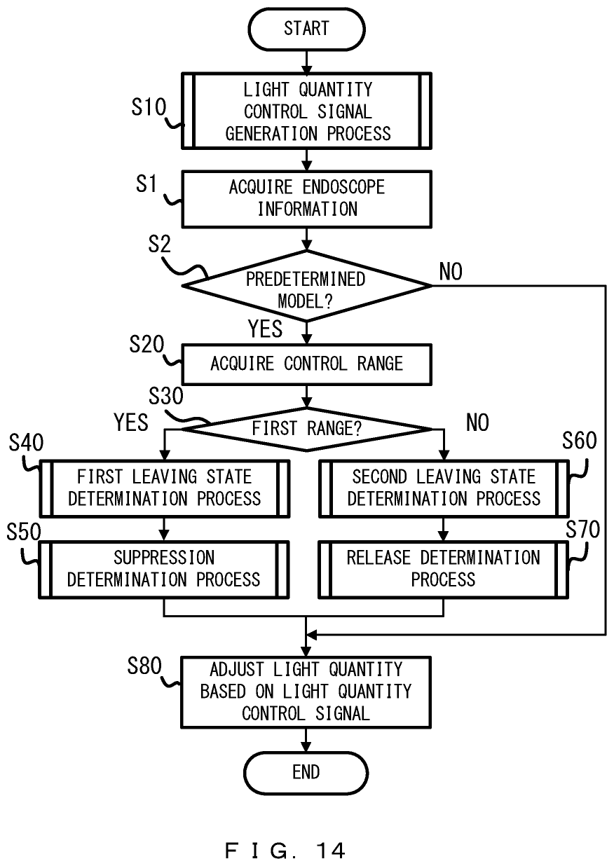

FIG. 14 is an example of a flowchart of a light quantity control process performed by the endoscope system 2. In the light quantity control process illustrated in FIG. 14, after the light quantity adjustment calculation unit 25 generates the light quantity control signal in step S10, the model identification unit 27 acquires the endoscope information (step S1), and determines whether the model of the endoscope 10 is a predetermined model based on the acquired endoscope information (step S2).

In step S2, the model identification unit 27 determines whether the model of the endoscope 10 is a model in which the thin insertion portion causes heat to easily stay therein, for example, a model for trachea and bronchus. The information of the predetermined model may be stored in the processor memory 21, for example.

When it is determined, in step S2, that the endoscope 10 is the predetermined model, the endoscope system 2 performs processing of steps S20 to S80. The processing of steps S20 to S80 is the same as the processing of steps S20 to S80 illustrated in FIG. 3. When it is not determined, in step S2, that the endoscope 10 is the predetermined model, the endoscope system 2 performs the processing of step S80 by skipping the processing of steps S20 to S70. That is, the endoscope system 2 performs the light quantity control without changing the control range of the illumination light quantity from the first range.

The light source control device or the endoscope system 2 according to the present embodiment can achieve the same effect as that achieved by the light source control device or the endoscope system 1 according to the first embodiment. Furthermore, according to the light source control device or the endoscope system 2 according to the present embodiment, the control range of the illumination light quantity can be adjusted only when the endoscope of the predetermined model in which the distal end is easily heated to the high temperature is used. This can further reduce the possibility that convenience of the operator is impaired when the control range is suppressed in an unnecessary case.

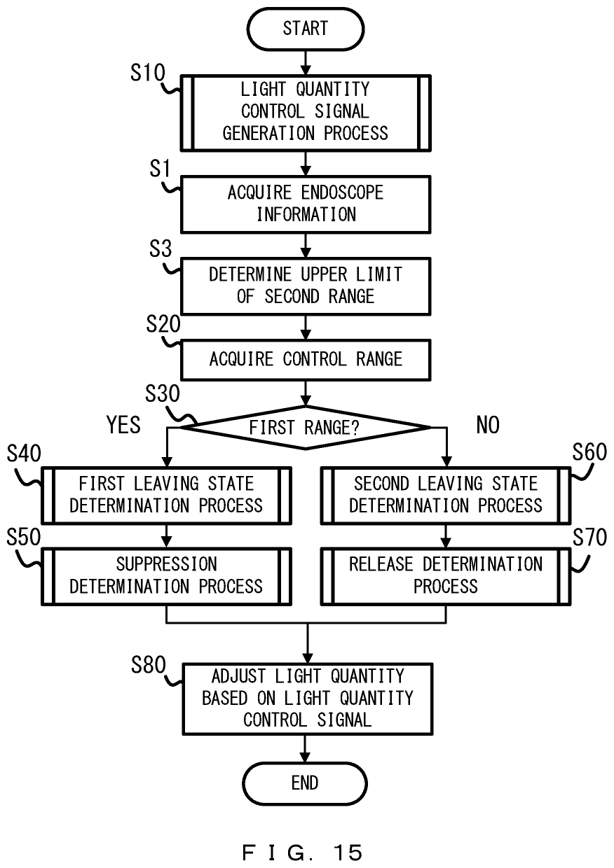

FIG. 15 is another example of a flowchart of a light quantity control process performed by the endoscope system 2. The endoscope system 2 may perform the light quantity control process illustrated in FIG. 15 instead of the light quantity control process illustrated in FIG. 14.

In the light quantity control process illustrated in FIG. 15, after the light quantity adjustment calculation unit 25 generates the light quantity control signal in step S10, the model identification unit 27 acquires the endoscope information (step S1), and determines the upper limit of the second range based on the acquired endoscope information (step S3).

In step S3, the model identification unit 27 identifies the model of the endoscope based on the endoscope information. Then, the model identification unit 27 determines the upper limit of the second range according to the identified model. The upper limit of the second range for each model may be stored in the processor memory 21, for example.

Then, the endoscope system 2 performs processing of steps S20 to S80. The processing of steps S20 to S80 is the same as the processing of steps S20 to S80 illustrated in FIG. 3.

The light source control device or the endoscope system 2 according to the present embodiment performs the light quantity control process illustrated in FIG. 15, thereby being able to achieving the same effect as that achieved by the light source control device or the endoscope system 1 according to the first embodiment. Furthermore, the light source control device or the endoscope system 2 according to the present embodiment performs the light quantity control process illustrated in FIG. 15, whereby the upper limit of the second range can be changed according to the model of the endoscope to be used. This enables the upper limit of the control range of the illumination light quantity to be limited to be low for the predetermined models in which the distal end is easily heated to the high temperature, for example. That is, the illumination light quantity can be limited in a necessary range according to the model of the endoscope.

In the above description, an example is shown in which the light source control device or the endoscope system 2 according to the present embodiment performs different controls according to the model of the endoscope, but may perform different control not according to the model of the endoscope but according to the endoscope. For example, the upper limit of the second range appropriate for the endoscope may be stored in advance in the endoscope memory 13 so that an upper limit of the second range can be changed according to the upper limit of the second range read from the endoscope memory 13. This enables the illumination range to be set in consideration of individual difference in the endoscope.

### Third Embodiment

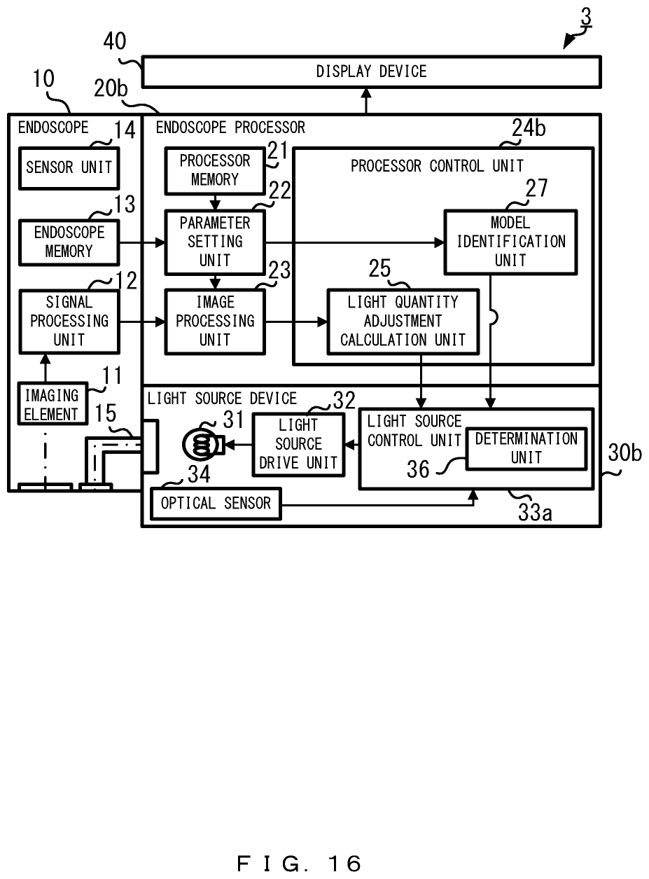

FIG. 16 is a diagram illustrating a configuration of an endoscope system 3 according to the present embodiment. The endoscope system 3 illustrated in FIG. 16 is different from the endoscope system 2 in that an endoscope processor 20b is provided instead of the endoscope processor 20a, and a light source device 30b is provided instead of the light source device 30. The other components are the same as those in the endoscope system 2.

The endoscope processor 20b is different from the endoscope processor 20a in that a processor control unit 24b including no determination unit 26 is provided, and the light source device 30b is different from the light source device 30 in that a light source control unit 33a including a determination unit 36 is provided. The determination unit 36 is a circuit that determines whether the endoscope 10 is left, at least based on the light quantity control signal, and is the same as the determination unit 26 of the endoscope system 2. That is, the endoscope system 3 is different from the endoscope system 2 in that the determination unit that determines whether the endoscope 10 is left is included not in the endoscope processor 20 but in the light source device 30.

The light source control device or the endoscope system 3 according to the present embodiment can achieve the same effect as that achieved by the light source control device or the endoscope system 2 according to the second embodiment.

### Fourth Embodiment

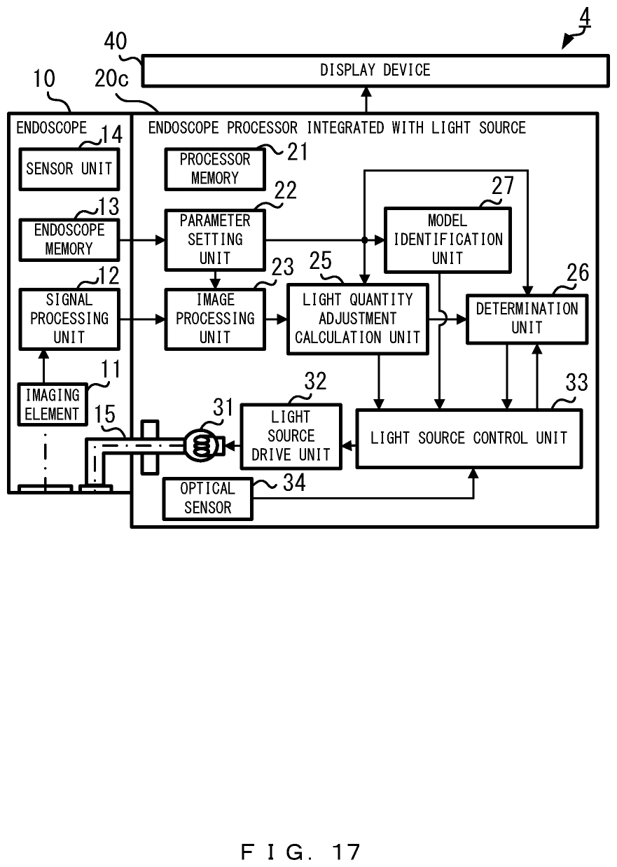

FIG. 17 is a diagram illustrating a configuration of an endoscope system 4 according to the present embodiment. The endoscope system 4 illustrated in FIG. 17 is different from the endoscope system 2 in that an endoscope processor 20c integrated with the light source device is provided instead of the endoscope processor 20a and the light source device 30. The other components are the same as those in the endoscope system 2. Note that the configuration of the endoscope processor 20c is the same as a combination of the configurations of the endoscope processor 20a and the light source device 30.

The light source control device or the endoscope system 4 according to the present embodiment can achieve the same effect as that achieved by the light source control device or the endoscope system 2 according to the second embodiment.

The above-described embodiments indicate specific examples to facilitate the understanding of the invention, and the embodiments of the present invention are not limited to these. The light source control device, the endoscope system, and the light quantity control method can be variously modified or changed without departing from the scope of the claims.

In the example described above, for example, the endoscope system and the light source control device are an endoscope system and a light source control device that are used for medical purpose. However, the endoscope system and the light source device are not limited to an endoscope system and a light source control device that are used for medical purpose. For example, similarly in the case of the endoscope system and the light source control device that are used for an industrial purpose, the distal end of the endoscope is heated to the high temperature unless the appropriate light quantity control is performed when the endoscope is left. Therefore, the same effect can be achieved by applying the above-described light quantity control. Furthermore, in the example described above, the endoscope is a flexible endoscope. However, the endoscope is not limited to the flexible endoscope. The endoscope may be, for example, a rigid endoscope.

As illustrated in FIG. 7, the condition for determining that the endoscope is not left is defined such that there is a change in the imaging signal, the endoscope is operated, or the like. However, it may be determined that the endoscope is not left, under the other conditions. For example, it may be determined that the endoscope is not left, under the condition in which a predetermined period of time elapses from when the control range is suppressed, there is a change in the illumination light quantity detected by the optical sensor 34, and the like, and the suppression of the control range may be released. Alternatively, it may be determined that the endoscope is not left by satisfying a combination of some of the above-described conditions, and the suppression may be released. Alternatively, when the operator explicitly directs to release the suppression of the control range, the suppression of the control range may be released.

In FIG. 5, an example is shown in which the light quantity control signal and the illumination light quantity are used for the condition for determining that the endoscope is left. However, it may be determined that the endoscope is left in combination with the other conditions. For example, the control range may be suppressed when the conditions are satisfied in which the above-described predetermined state is maintained for a predetermined period of time, and furthermore there is no change in image, there is no endoscope operation, and the like. Alternatively, when the operator explicitly directs to suppress the control range, the control range may be suppressed.

In FIGS. 1, 13, 16, and 17, an example is shown in which the endoscope processor or the light source device determines whether the endoscope 10 is left. However, the determination may be made by the endoscope 10 that has received the light quantity control signal from the endoscope processor.

In this specification, the expression “based on A” does not mean “based on only A,” but means “at least based on A,” and furthermore means “based at least in part on A.” That is, “based on A” may mean “based on B in addition to A” and may mean “based in part on A.”

What is claimed is:

1. A light source control device for an endoscope, comprising:
one or more processors that perform processes,
the processes including:
generating a light quantity control signal indicating excess or insufficiency of an illumination light quantity supplied from the light source control device to the endoscope based on an imaging signal from an imaging element of the endoscope;
determining whether the endoscope is left based on the light quantity control signal and whether the illumination light quantity is equal to or greater than a predetermined quantity; and
setting a control range that limits the illumination light quantity to be supplied from the light source control device to the endoscope based on the light quantity control signal, wherein
the setting the control range includes setting a second range having an upper limit lower than an upper limit of a first range as the control range, when the one or more processors determine that the endoscope is left in a state in which the first range is set as the control range.

2. The light source control device according to claim 1, wherein
the determining whether the endoscope is left includes:
determining whether a state in which the illumination light quantity supplied or to be supplied from the light source control device to the endoscope is equal to or greater than the predetermined quantity and the light quantity control signal indicating insufficiency of the illumination light quantity is generated is maintained for a predetermined period of time or longer, when the first range is set as the control range; and
determining that the endoscope is left, when it is determined that the state is maintained for the predetermined period of time or longer.

3. The light source control device according to claim 2, wherein
the predetermined quantity is an illumination light quantity corresponding to an upper limit of the first range.

4. The light source control device according to claim 1, further comprising:
a light source that emits illumination light to be supplied to the endoscope; and
an optical sensor that measures an illumination light quantity emitted from the light source,
wherein the processes further include:
generating information on the illumination light quantity supplied from the light source control device to the endoscope based on the illumination light quantity measured by the optical sensor.

5. The light source control device according to claim 1, further comprising:
a light source that emits illumination light to be supplied to the endoscope; and
a light source drive unit that drives the light source,
wherein the processes further include:
generating information on the illumination light quantity to be supplied from the light source control device to the endoscope based on the illumination light quantity directed to the light source drive unit.

6. The light source control device according to claim 1, wherein
the setting the control range further includes setting the first range as the control range when the one or more processors determine that the endoscope is not left when the second range is set as the control range.

7. The light source control device according to claim 6, wherein
the determining whether the endoscope is left includes:
determining that the endoscope is left according to criteria more stringent than those when the first range is set as the control range, when the second range is set as the control range.

8. The light source control device according to claim 6, wherein
the determining whether the endoscope is left includes:
determining whether a state in which the illumination light quantity is equal to or greater than a predetermined quantity and the light quantity control signal indicating insufficiency of the illumination light quantity is generated is maintained for a predetermined period of time or longer, when the second range is set as the control range; and
determining that the endoscope is not left, when it is not determined that the state is maintained for the predetermined period of time or longer.

9. The light source control device according to claim 6, wherein
the processes further include processing the imaging signal, and
the determining whether the endoscope is left includes:
determining whether a change in the imaging signal has been detected by processing the imaging signal, when the second range is set as the control range; and
determining that the endoscope is not left when it is determined that a change in the imaging signal has been detected by processing the imaging signal.

10. The light source control device according to claim 6, wherein
the determining whether the endoscope is left includes:
determining whether a sensor unit of the endoscope has detected an operation on the endoscope, when the second range is set as the control range; and
determining that the endoscope is not left when it is determined that the sensor unit has detected an operation on the endoscope.

11. The light source control device according to claim 1, wherein
the generating of the light quantity control signal includes generating the light quantity control signal based on at least an evaluation value of brightness of an image calculated from the imaging signal, and a target value of the brightness of the image.

12. The light source control device according to claim 1, wherein
the processes further include
identifying a model of the endoscope based on information of the endoscope read from a memory included in the endoscope, and
the setting the control range includes:
setting the second range as the control range when the one or more processors determine that the endoscope is left in a state in which the model of the endoscope is identified to be a predetermined model by identifying the model of the endoscope and the first range is set as the control range; and
not setting the second range as the control range when the model of the endoscope is identified to be a model other than the predetermined model by identifying the model of the endoscope.

13. The light source control device according to claim 1, wherein
the processes further include
identifying a model of the endoscope based on information of the endoscope read from a memory included in the endoscope, and
the setting the control range includes
determining an upper limit of the second range according to the model of the endoscope identified by identifying the model of the endoscope.

14. The light source control device according to claim 1, wherein
the setting the control range includes
determining an upper limit of the second range based on information read from a memory included in the endoscope.

15. The light source control device according to claim 1, further comprising:
a plurality of light sources that emit illumination light in different wavelength regions,
wherein the setting the control range includes
setting the control range to maintain a light quantity ratio of the illumination light emitted from the plurality of light sources, between a case where the first range is set as the control range and a case where the second range is set as the control range.

16. A light source control device for an endoscope, comprising:
one or more processors that perform processes, the processes including:
generating a light quantity control signal indicating excess or insufficiency of an illumination light quantity supplied from the light source control device to the endoscope based on an imaging signal from an imaging element of the endoscope;
determining whether the endoscope is left based on the light quantity control signal and whether the illumination light quantity is equal to or greater than a predetermined quantity; and
setting a control range that limits the illumination light quantity to be supplied from the light source control device to the endoscope based on the light quantity control signal, wherein
the setting the control range includes setting a first range having an upper limit higher than an upper limit of a second range as the control range, when the one or more processors determine that the endoscope is not left in a state in which the second range is set as the control range.

17. An endoscope system, comprising:
an endoscope;
a light source control device including one or more processors that perform processes,
the processes including:
generating a light quantity control signal indicating excess or insufficiency of an illumination light quantity supplied from the light source control device to the endoscope based on an imaging signal from an imaging element of the endoscope;
determining whether the endoscope is left based on the light quantity control signal and whether the illumination light quantity is equal to or greater than a predetermined quantity; and
setting a control range that limits the illumination light quantity to be supplied from the light source control device to the endoscope based on the light quantity control signal, wherein
the setting the control range includes setting a second range having an upper limit lower than an upper limit of a first range as the control range, when the one or more processors determine that the endoscope is left in a state in which the first range is set as the control range; and
a display device that displays a previous notification screen for previously notifying of a change of the control range from the first range to the second range or a screen showing that the illumination light quantity is suppressed during a period in which the second range is set.

18. A light quantity control method for a light source control device for an endoscope, the method comprising:
generating a light quantity control signal indicating excess or insufficiency of an illumination light quantity supplied from the light source control device to the endoscope based on an imaging signal from an imaging element of the endoscope;
determining whether the endoscope is left based on the light quantity control signal and whether the illumination light quantity is equal to or greater than a predetermined quantity;
setting a control range that limits the illumination light quantity to be supplied from the light source control device to the endoscope based on the light quantity control signal; and
setting a second range having an upper limit lower than an upper limit of a first range as the control range, when it is determined that the endoscope is left in a state in which the first range is set as the control range.
